(19) United States
(12) Patent Application Publication
(10) Pub. No.: US 2024/0366824 A1
(43) Pub. Date: Nov. 7, 2024

(54) SPACE PURIFICATION DEVICE

(71) Applicant: Panasonic Intellectual Property Management Co., Ltd., Kadoma-shi, Osaka (JP)

(72) Inventor: Fumiaki ASAI, Aichi (JP)

(21) Appl. No.: 18/688,821

(22) PCT Filed: Jul. 27, 2022
(86) PCT No.: PCT/JP2022/028981

§ 371 Date: Mar. 4, 2024

(30) Foreign Application Priority Data
Sep. 7, 2021 (JP) 2021-145173

(51) Int. Cl. A61L 9/14; A61L 101/06; A61L 101/20; C02F 1/461

(52) U.S. Cl. CPC A61L 9/145; C02F 1/46104; A61L 2101/06; A61L 2101/20; A61L 2209/11; A61L 2209/134; A61L 2209/213

(57) ABSTRACT

Space purification device (1000) of the present disclosure includes an electrolytic bath, electrode part (210), a mixing bath, hypochlorous acid water supply part (228), water supply part (138), an air blower, purification part (310), and controller (500). Controller (500) includes count part (550) that counts an elapsed time from timing at which generation of hypochlorous acid water of a first concentration in the electrolytic bath is completed, and calculation part (560) that calculates, based on a count time counted by count part (550), a concentration decrease value indicating how much the first concentration of the hypochlorous acid water in the electrolytic bath is decreased. Controller (500) is configured to adjust a supply amount of hypochlorous acid water to be supplied from hypochlorous acid water supply part (228) based on the concentration decrease value.

### TECHNICAL FIELD

The present disclosure relates to a space purification device that releases active oxygen species into a space.

### BACKGROUND ART

In order to remove (including inactivate) bacteria, fungi, viruses, odors, and the like in air, a space purification device that generates hypochlorous acid water by electrolysis and releases the hypochlorous acid water is known (see, for example, PTL 1). Generating hypochlorous acid requires inputting an electrolysis accelerator such as salt to water to generate water containing chloride ions.

### CITATION LIST

### Patent Literature

PTL 1: Unexamined Japanese Patent Publication No. 2019-24811

### SUMMARY OF THE INVENTION

In a conventional space purification device, hypochlorous acid water containing hypochlorous acid which is one of active oxygen species is generated in a water storage part, the generated hypochlorous acid water and air sucked from outside are continuously brought into contact with each other inside, and the air brought into contact is directly released from the water storage part to the outside. When hypochlorous acid water stored in the water storage part is contaminated by dust, PM2.5, or the like due to contact between the hypochlorous acid water and the air sucked from the outside, electrodes might be deteriorated. Therefore, it is conceivable to separately provide an electrolytic bath in which hypochlorous acid water is generated and a mixing bath in which the hypochlorous acid water in the electrolytic bath and water are mixed.

By bringing hypochlorous acid water in the mixing bath into contact with air sucked from the outside and not bringing air sucked from the outside into contact with the electrolytic bath in which the electrodes are present, deterioration of an electrode part can be suppressed. On this occasion, an amount of hypochlorous acid in the mixing bath is determined by a supply amount of hypochlorous acid water from the electrolytic bath to the mixing bath. However, it was found that after generation of hypochlorous acid water having a predetermined concentration in the electrolytic bath, hypochlorous acid in the electrolytic bath decreased and the concentration of the hypochlorous acid water in the electrolytic bath decreased as time elapsed in a state where the generated hypochlorous acid water and the electrode part were in contact with each other.

An object of the present disclosure is to optimize a supply amount of hypochlorous acid water based on a decrease in concentration.

A space purification device according to an aspect of the present disclosure includes: an electrolytic bath that stores water supplied from outside; an electrode part that is provided in the electrolytic bath and generates hypochlorous acid water having a first concentration from the water stored in the electrolytic bath; a mixing bath that mixes the hypochlorous acid water and water to generate mixed water; a hypochlorous acid water supply part that supplies the hypochlorous acid water from the electrolytic bath to the mixing bath; a water supply part that supplies the water to the mixing bath; an air blower that takes in air from an air suction port based on preset air volume setting; a purification part that brings the mixed water stored in the mixing bath into contact with the air sucked from the air suction port; and a controller that controls the electrode part, the hypochlorous acid water supply part, the water supply part, and the air blower. The controller includes a count part that counts an elapsed time from timing at which generation of the hypochlorous acid water having the first concentration in the electrolytic bath is completed, and a calculation part that calculates, based on a count time counted by the count part, a concentration decrease value indicating how much the first concentration of the hypochlorous acid water in the electrolytic bath is decreased. The controller is configured to adjust a supply amount of the hypochlorous acid water to be supplied from the hypochlorous acid water supply part based on the concentration decrease value.

Any combinations of the above-described components, and conversion of the expressions of the present disclosure into methods, devices, systems, recording media, or computer programs are also effective as modes of the present disclosure.

According to the present disclosure, it is possible to provide a space purification device that stably realizes disinfection and deodorization performance.

### BRIEF DESCRIPTION OF THE DRAWINGS

FIG. 1 is a view illustrating an internal configuration of a space purification device according to an exemplary embodiment of the present disclosure.

FIG. 2 is a diagram illustrating functional blocks of the space purification device according to the exemplary embodiment of the present disclosure.

FIG. 3A is a diagram showing a data structure of a table stored in a storage part of the space purification device according to the exemplary embodiment of the present disclosure.

FIG. 3B is a diagram showing a data structure of a table stored in the storage part of the space purification device according to the exemplary embodiment of the present disclosure.

FIG. 4A is a diagram showing a data structure of another table stored in the storage part of the space purification device according to the exemplary embodiment of the present disclosure.

FIG. 4B is a diagram showing a data structure of a further table stored in the storage part of the space purification device according to the exemplary embodiment of the present disclosure.

FIG. 5 is a flowchart showing a control procedure by the space purification device according to the exemplary embodiment of the present disclosure.

### DESCRIPTION OF EMBODIMENT

The present exemplary embodiment relates to a space purification device that generates hypochlorous acid water based on water and an electrolysis accelerator and then releases the hypochlorous acid water. In a conventional space purification device, hypochlorous acid water containing active oxygen species is generated in a water storage part. Also in the conventional space purification device, generated hypochlorous acid water and air sucked from outside are continuously brought into contact with each other in the water storage part, and then, the air brought into contact is released to the outside by rotation of a fan as an air blower. Therefore, the hypochlorous acid water in the water storage part is easily contaminated due to contact with air. When the hypochlorous acid water is contaminated, electrodes might be deteriorated.

In order to suppress deterioration of electrodes, in the space purification device according to the present exemplary embodiment, a conventional water storage part is divided into two water tanks, an electrolytic bath and a mixing bath. The electrolytic bath is provided with electrodes, and the electrodes electrolyze water containing chloride ions to generate hypochlorous acid water in the electrolytic bath. The hypochlorous acid water generated in the electrolytic bath is supplied to the mixing bath. Furthermore, in the mixing bath, the hypochlorous acid water from the electrolytic bath and air sucked from outside are continuously brought into contact with each other, and then, the air brought into contact is released to the outside by rotation of a fan. With such configuration, the hypochlorous acid water in the electrolytic bath does not come into contact with air, making the hypochlorous acid water less likely to be contaminated, and suppressing the electrodes from deteriorating.

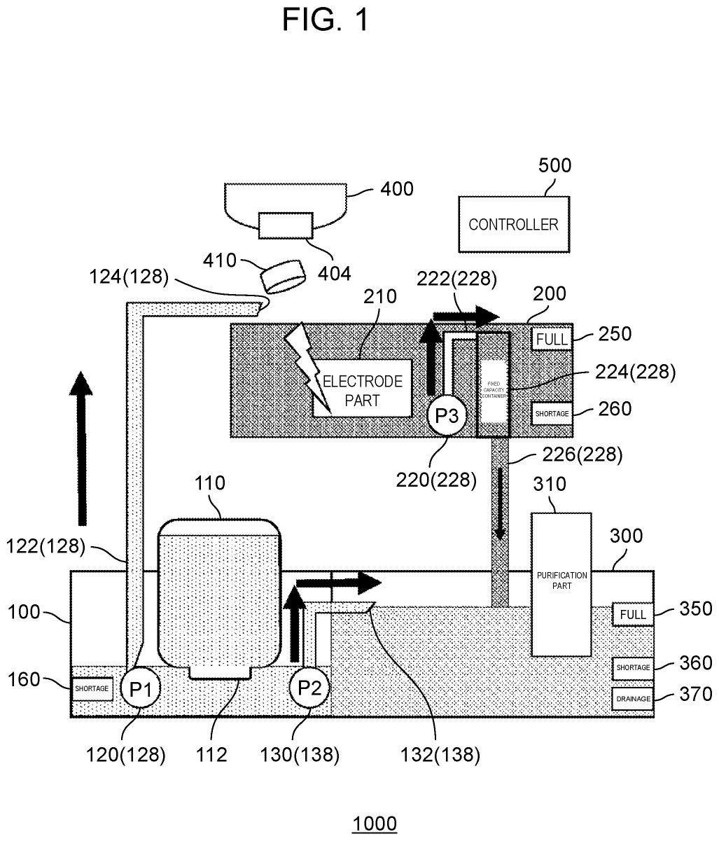

In the following, a mode for carrying out the present disclosure will now be described with reference to the accompanying drawings. FIG. 1 is a view illustrating an internal configuration of space purification device 1000 according to an exemplary embodiment of the present disclosure.

Space purification device 1000 includes water storage tank 100, water supply tank 110, lid 112, first pump 120, first water supply pipe 122, supply port 124, second pump 130, second water supply pipe 132, water shortage float 160, electrolytic bath 200, electrode part 210, third pump 220, third water supply pipe 222, fixed capacity container 224, third water supply pipe 226, full water float 250, water shortage float 260, mixing bath 300, purification part 310, full water float 350, water shortage float 360, drainage float 370, electrolysis accelerator input part 400, input port 404, electrolysis accelerator 410, and controller 500. Here, first pump 120, first water supply pipe 122, and supply port 124 are included in first supply part 128, second pump 130 and second water supply pipe 132 are included in second supply part 138, and third pump 220, third water supply pipe 222, fixed capacity container 224, and third water supply pipe 226 are included in third supply part 228. In the following, (1) basic configuration, (2) hypochlorous acid water generation processing, (3) reprocessing, and (4) water feeding processing will be described in this order.

### (1) Basic Configuration

Water storage tank 100 has a box shape with a top face opened, and has a structure enabling storage of water to store water supplied from water supply tank 110 to be described later. Water storage tank 100 is disposed in, for example, a lower part of space purification device 1000. Water supply tank 110 is a tank that internally stores water, and is detachable from water storage tank 100. An opening (not illustrated) of water supply tank 110 is provided with lid 112, and at a center of lid 112, an opening and closing part (not illustrated) is provided. When the opening and closing part is opened, water in water supply tank 110 is supplied to water storage tank 100.

Specifically, when water supply tank 110 is attached to water storage tank 100 with the opening of water supply tank 110 facing downward, the opening and closing part is opened. In other words, when water supply tank 110 containing water is attached to water storage tank 100, the opening and closing part is opened to supply water to water storage tank 100, and the water collects in water storage tank 100. When a water level in water storage tank 100 rises and reaches lid 112, the water supply is stopped because the opening of water supply tank 110 is water-sealed. When water remains inside water supply tank 110, the water in water supply tank 110 is supplied to water storage tank 100 every time the water level in water storage tank 100 drops. As a result, the water level in water storage tank 100 is kept constant.

First pump 120 is disposed in water storage tank 100, and is connected to first water supply pipe 122. When operating in response to an instruction from controller 500, first pump 120 pumps up water stored in water storage tank 100 toward first water supply pipe 122. First water supply pipe 122 is a pipe connecting water storage tank 100 and electrolytic bath 200, and has supply port 124 at an end on electrolytic bath 200 side. The water pumped up by first pump 120 flows in first water supply pipe 122, and is supplied from supply port 124 to electrolytic bath 200. In other words, first pump 120, first water supply pipe 122, and supply port 124 supply water from water storage tank 100 to electrolytic bath 200.

Second pump 130 is disposed in water storage tank 100, and is connected to second water supply pipe 132. When operating in response to an instruction from controller 500, second pump 130 pumps up water stored in water storage tank 100 toward second water supply pipe 132. Second water supply pipe 132 is a pipe connecting water storage tank 100 and mixing bath 300. The water pumped up by second pump 130 flows in second water supply pipe 132, and is supplied to mixing bath 300. In other words, second pump 130 and second water supply pipe 132 can be said to be water supply parts that supply water from water storage tank 100 to mixing bath 300.

Electrolytic bath 200 has a box shape with a top face opened, and is disposed below supply port 124. Electrolytic bath 200 stores water supplied from supply port 124. Above electrolytic bath 200, electrolysis accelerator input part 400 is disposed side by side with supply port 124.

Electrolysis accelerator input part 400 can be internally loaded with electrolysis accelerator 410, and rotates a tablet input member (not illustrated) upon receiving an input instruction of electrolysis accelerator 410 from controller 500. When the tablet input member rotates, electrolysis accelerator 410 drops into electrolytic bath 200. Electrolysis accelerator input part 400 counts the number of electrolysis accelerators 410 dropped into electrolytic bath 200, and stops the rotation of the tablet input member upon determining that one tablet of electrolysis accelerator 410 has dropped into electrolytic bath 200. In other words, electrolysis accelerator input part 400 inputs electrolysis accelerator 410 into electrolytic bath 200. When electrolysis accelerator 410 dissolves into water in electrolytic bath 200, water containing chloride ions is generated in electrolytic bath 200. One example of electrolysis accelerator 410 is sodium chloride and is formed as an electrolysis acceleration tablet.

Electrode part 210 is installed in a manner to be immersed in water in electrolytic bath 200. By being electrically conducted, electrode part 210 electrochemically electrolyzes water containing chloride ions in electrolytic bath 200 to generate electrolyzed water containing active oxygen species. Here, the active oxygen species represents oxygen molecules having an oxidation activity higher than an oxidation activity of normal oxygen, and substances related thereto. For example, in addition to what is called a narrow sense of active oxygen such as superoxide anion, singlet oxygen, hydroxyl radical, or hydrogen peroxide, the active oxygen species include what is called a broad sense of active oxygen such as ozone or hypochlorous acid (hypohalous acid). In the present exemplary embodiment, hypochlorous acid water is generated as electrolyzed water.

Electrode part 210 may generate hypochlorous acid water by repeating one cycle a plurality of times, one cycle having electric conduction time for performing electric conduction for electrolysis and time after stopping the electric conduction, i.e., non-electric conduction time that is time when the electric conduction is not performed. By providing the non-electric conduction time for electrode part 210, a life of electrode part 210 is extended. When the electric conduction time is lengthened with respect to the non-electric conduction time, hypochlorous acid water containing a larger amount of active oxygen species is generated per cycle. When the non-electric conduction time is lengthened with respect to the electric conduction time, generation of active oxygen species per cycle can be suppressed. Furthermore, when an amount of electricity in the electric conduction time is increased, hypochlorous acid water containing a larger amount of active oxygen species is generated. As described above, it can be said that electrolytic bath 200 is a tank for generating hypochlorous acid water from water into which electrolysis accelerator 410 has been input. Hypochlorous acid water having a predetermined concentration is generated in electrolytic bath 200.

Third pump 220 is disposed in electrolytic bath 200, and is connected to third water supply pipe 222. When operating in response to an instruction from controller 500, third pump 220 pumps up hypochlorous acid water stored in electrolytic bath 200 toward third water supply pipe 222. Third water supply pipe 222 is connected to fixed capacity container 224, and supplies the hypochlorous acid water in electrolytic bath 200 to fixed capacity container 224. Fixed capacity container 224 is a measuring container having a fixed capacity, and stores hypochlorous acid water supplied from third water supply pipe 222. Fixed capacity container 224 is connected to third water supply pipe 226, and third water supply pipe 226 extends toward mixing bath 300. Hypochlorous acid water stored in fixed capacity container 224 flows in third water supply pipe 226 and is supplied to mixing bath 300. In other words, third pump 220, third water supply pipe 222, fixed capacity container 224, and third water supply pipe 226 supply hypochlorous acid water from electrolytic bath 200 to mixing bath 300.

Mixing bath 300 has a box shape with a top face opened, and mixes water supplied from water storage tank 100 and hypochlorous acid water supplied from electrolytic bath 200 to generate mixed water. This corresponds to diluting the hypochlorous acid water supplied from electrolytic bath 200 with the water supplied from water storage tank 100. Mixing bath 300 is provided with purification part 310.

Purification part 310 includes air blower 518 and a filter (not illustrated). An example of air blower 518 is, for example, a fan, and rotates under the control of controller 500. Air blower 518 operates with preset air volume setting. A rotation speed of the fan is controlled based on the set air volume. As the fan rotates, air is taken into space purification device 1000 from an air suction port (not illustrated) provided in a casing (not illustrated) of space purification device 1000.

The filter is a member that brings the hypochlorous acid water stored in mixing bath 300 into contact with indoor air having flowed into space purification device 1000 by air blower 518. The hypochlorous acid water stored in mixing bath 300 can be also said to be mixed water of hypochlorous acid water and water. The filter is formed in a cylindrical shape, and has a circumferential part where holes through which air can flow are provided. The filter is rotatably incorporated in mixing bath 300 with a central axis as a rotation center so that one end of the filter is immersed in the hypochlorous acid water stored in mixing bath 300 to retain water. The filter is rotated by a driving part (not illustrated) to bring the hypochlorous acid water and the indoor air into continuous contact with each other.

An air path leading from the air suction port to the filter, the fan, and a blow-out port (not illustrated) is formed. When the fan rotates, external air having been sucked from the air suction port and having entered the air path is blown out to the outside of space purification device 1000 sequentially via the filter, the fan, and the blow-out port. As a result, the air brought into contact with the hypochlorous acid water in mixing bath 300 is released to the outside. Space purification device 1000 releases active oxygen species derived from the hypochlorous acid water having been generated (including volatilization) together with the air.

Water shortage float 160 provided in water storage tank 100, full water float 250 and water shortage float 260 provided in electrolytic bath 200, and full water float 350, water shortage float 360, and drainage float 370 provided in mixing bath 300 each detects whether or not water or hypochlorous acid water is present. Here, water and hypochlorous acid water may be collectively referred to as “water”. Water shortage float 160, full water float 250, water shortage float 260, full water float 350, water shortage float 360, and drainage float 370 are collectively referred to as “float”. Each of the floats has buoyancy and further has a magnet (not illustrated), and a position of the magnet is detected by a detection part (not illustrated). In a case where water is present up to a position of the float, the float moves to a predetermined position by buoyancy, and the detection part detects the magnet provided on the float part. On the other hand, in a case where no water is present up to the position of the float, the detection part cannot detect the magnet provided on the float.

Water shortage float 160 detects water shortage of water storage tank 100. Full water float 250 detects full water of electrolytic bath 200. Water shortage float 260 detects water shortage of electrolytic bath 200. In addition, full water float 350 detects full water of mixing bath 300 and water shortage float 360 detects water shortage of mixing bath 300. Drainage float 370 detects a drainage level of mixing bath 300. Here, the water shortage does not have to be a 100% water shortage, and allows a slight amount of water to remain. In addition, the full water does not have to be 100% full water, and may be a water amount at which water can be further input. Each float transmits a detection result to controller 500.

Controller 500 receives detection results from water shortage float 160, full water float 250, water shortage float 260, full water float 350, water shortage float 360, and drainage float 370. Controller 500 executes control of electrode part 210, purification part 310, electrolysis accelerator input part 400, first supply part 128, second supply part 138, and third supply part 228. Details of processing of controller 500 will be described later.

As an example, a concentration of hypochlorous acid water to be generated in electrolytic bath 200 is a concentration within a range of 30 ppm to 200 ppm (hereinafter, referred to as “first concentration”), and a concentration of hypochlorous acid water to be diluted in mixing bath 300 is a predetermined concentration within a range of 3 ppm to 50 ppm. The concentration of the hypochlorous acid water to be diluted in mixing bath 300 is set to be lower than the concentration of the hypochlorous acid water to be generated in electrolytic bath 200.

### (2) Hypochlorous Acid Water Generation Processing

The hypochlorous acid water generation processing is processing for generating hypochlorous acid water having the first concentration in electrolytic bath 200.

Here, an example of a flow of the hypochlorous acid water generation processing will be described. A user pours water into water supply tank 110, and attaches water supply tank 110 to water storage tank 100. When water supply tank 110 is attached to water storage tank 100, water is supplied from water supply tank 110 to water storage tank 100 as a result of opening of the opening and closing part of lid 112.

By operating second pump 130, controller 500 supplies water in water storage tank 100 to mixing bath 300. The water is supplied until full water float 350 detects full water. As a result, mixing bath 300 stores water in a state of being full water.

By operating first pump 120, controller 500 supplies water in water storage tank 100 to electrolytic bath 200. On this occasion, water is supplied for a certain period of time for which electrolytic bath 200 does not become full of water. As a result of the water supply, a water surface of electrolytic bath 200 is present at a water level lower than a water level of full water. After the water supply is ended, controller 500 causes electrolysis accelerator 410 to be dropped from input port 404 toward electrolytic bath 200. As a result, electrolysis accelerator 410 begins to dissolve in water.

Subsequently, by operating first pump 120 again, controller 500 supplies water in water storage tank 100 to electrolytic bath 200. On this occasion, a pressure of the supplied water further promotes the dissolution of electrolysis accelerator 410. The water is supplied until full water float 250 detects full water. As a result, mixing bath 300 stores water containing chloride ions in a state of full water.

By executing electric conduction to electrode part 210, controller 500 electrolyzes the water containing chloride ions to generate hypochlorous acid water. Here, electrolysis time is time (e.g., 40 minutes) required for generating hypochlorous acid water having the first concentration. As a result, the hypochlorous acid water having the first concentration is generated. The foregoing is a flow of the hypochlorous acid water generation processing.

When the hypochlorous acid water having the first concentration is generated, controller 500 supplies the hypochlorous acid water having the first concentration to mixing bath 300 by operating third pump 220. On this occasion, controller 500 controls an amount of the hypochlorous acid water to be supplied to mixing bath 300, and details of the control will be described later. The hypochlorous acid water having the first concentration is diluted in mixing bath 300. By stopping third pump 220 and then operating purification part 310, controller 500 releases the air brought into contact with the hypochlorous acid water in mixing bath 300 to the outside of space purification device 1000.

### (3) Reprocessing

The reprocessing is processing for executing the hypochlorous acid generation processing again when water shortage float 260 detects water shortage, i.e., when the hypochlorous acid water in electrolytic bath 200 becomes short.

When the air brought into contact with the hypochlorous acid water is released, the amount of the hypochlorous acid water in mixing bath 300 decreases. Although to be described in detail later, controller 500 determines supply timing and a supply amount of the hypochlorous acid water to be supplied to mixing bath 300, and supplies the hypochlorous acid water to mixing bath 300 by operating third pump 220. When mixing bath 300 is not full of water, controller 500 further operates second pump 130 to supply water in water storage tank 100 to mixing bath 300 until mixing bath 300 becomes full of water. This causes release of the hypochlorous acid water to be continued. Such processing is repeated until water shortage float 260 detects water shortage.

When water shortage float 260 detects water shortage, the reprocessing is executed. Controller 500 starts the supply of water to electrolytic bath 200 by first supply part 128. In other words, controller 500 does not supply water to electrolytic bath 200 until electrolytic bath 200 becomes short of water. This is also for making impurities such as inorganic salt compounds be less likely to remain in electrolytic bath 200 by making old hypochlorous acid water hardly remain in electrolytic bath 200. As a result, a maintenance frequency of electrolytic bath 200 is reduced.

Here, similarly to the hypochlorous acid generation processing, controller 500 executes water supply for a certain period of time for which electrolytic bath 200 is not filled to full water. Subsequently, controller 500 causes electrolysis accelerator 410 to be dropped from input port 404 toward electrolytic bath 200, and executes water supply until electrolytic bath 200 becomes full of water. By executing electric conduction to electrode part 210, controller 500 also generates hypochlorous acid water having the first concentration, and then supplies the hypochlorous acid water having the first concentration from electrolytic bath 200 to mixing bath 300. In other words, the same processing as a part of the hypochlorous acid water generation processing is executed.

### (4) Water Feeding Processing

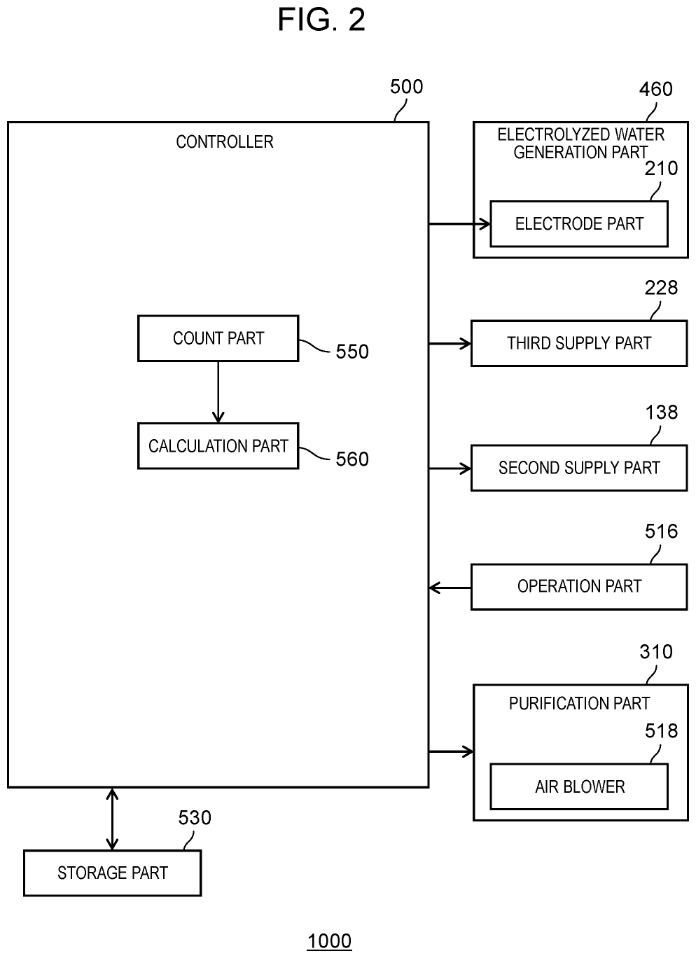

FIG. 2 illustrates functional blocks of space purification device 1000. Space purification device 1000 includes second supply part 138, third supply part 228, purification part 310, electrolyzed water generation part 460, controller 500, operation part 516, and storage part 530. Purification part 310 includes air blower 518, electrolyzed water generation part 460 includes electrode part 210, and controller 500 includes count part 550 and calculation part 560.

The water feeding processing is processing for operating third pump 220 of third supply part 228 in order to supply the hypochlorous acid water in electrolytic bath 200 to mixing bath 300. Third supply part 228 is also referred to as a hypochlorous acid water supply part, and the hypochlorous acid water supply part supplies hypochlorous acid water from electrolytic bath 200 to mixing bath 300. In order to optimize a concentration of the hypochlorous acid water in mixing bath 300, the water feeding processing includes (4-1) supply timing control and (4-2) supply amount control.

### (4-1) Supply Timing Control

In the supply timing control, supply of hypochlorous acid water from the electrolytic bath 200 to mixing bath 300 is determined when either (4-1-1) a first condition or (4-1-2) a second condition is satisfied.

### (4-1-1) First Condition

Controller 500 calculates a consumption amount of hypochlorous acid (active oxygen species) from after completion of supply of the previous hypochlorous acid water, and determines the supply of the hypochlorous acid water from electrolytic bath 200 to mixing bath 300 when the first condition is satisfied that an integrated value of the consumption amounts of hypochlorous acid is equal to or larger than a specified value.

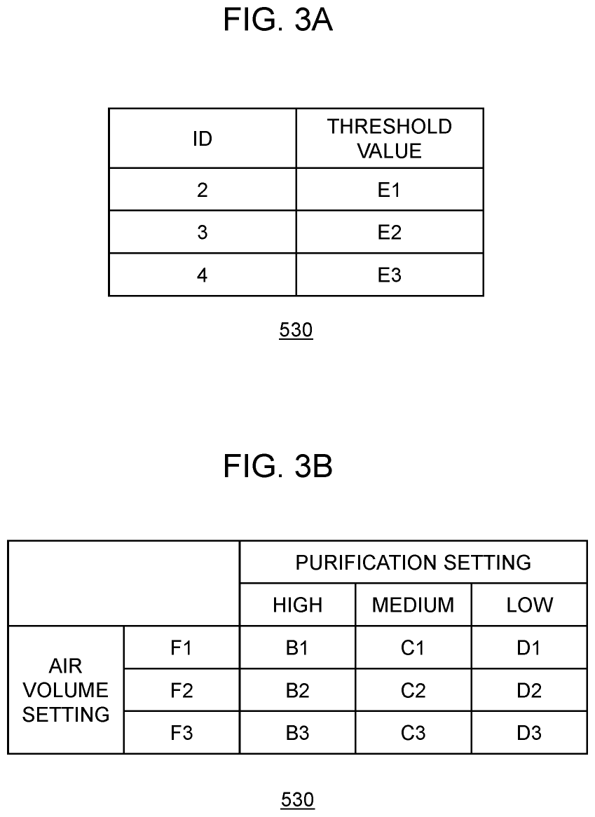

FIGS. 3A and 3B show data structures of tables stored in storage part 530. FIG. 3A illustrates a table for determining a threshold value. “ID” is identification information for identifying a supply amount. As an example, “ID” may take 2, 3, and 4. “ID=2” corresponds to a state in which a supply amount of hypochlorous acid is the smallest, and “ID=4” corresponds to a state in which the supply amount of hypochlorous acid is the largest. After the power of space purification device 1000 is initially turned on, controller 500 selects a threshold value corresponding to “ID=2”. On the other hand, other than the above, controller 500 selects a threshold value corresponding to the “ID” selected at the time of the previous supply. The threshold value corresponds to a supply amount of the hypochlorous acid at previous timing. FIG. 3B will be described later, and the description returns to FIG. 2.

Operation part 516 is an interface such as a button, or a touch panel, and accepts user's operation. Operation part 516 receives purification setting in purification part 310 as the user's operation. The purification setting is an index indicating a purification capability required of purification part 310. For example, the purification setting is indicated in three stages of “high”, “medium”, and “low”. The purification setting “high” is a state in which the required purification capability is the highest, and an amount of hypochlorous acid (active oxygen species) in air released from purification part 310 is set to be the highest. The purification setting “low” is a state in which the required purification capability is the lowest, and the amount of the hypochlorous acid (active oxygen species) in the air released from purification part 310 is set to be the lowest. Operation part 516 outputs the received purification setting to controller 500.

Furthermore, operation part 516 receives the air volume setting to be set to air blower 518 as user's operation. The air volume setting is setting of the air volume of air blower 518, i.e., the rotation speed of the fan. For example, the air volume setting is indicated in three stages of “F3”, “F2”, and “F1”. Air volume setting “F3” has the largest air volume and the highest rotation speed of the fan. Air volume setting “F1” has the smallest air volume and the lowest rotation speed of the fan. Operation part 516 outputs the received air volume setting to controller 500. In other words, controller 500 receives the purification setting and the air volume setting from operation part 516.

Next, a method of calculating a consumption amount of hypochlorous acid (active oxygen species) by controller 500 will be described. Controller 500 calculates a consumption amount of hypochlorous acid per unit time based on air volume setting and purification setting. FIG. 3B is a table showing consumption amounts of hypochlorous acid associated with air volume setting “F1” to “F3” and the purification setting “high”, “medium”, and “low”. For example, when the air volume setting is “F1” and the purification setting is “high”, the consumption amount of the hypochlorous acid is indicated as “B1”. The higher the air volume setting is, the larger the consumption amount of the hypochlorous acid per unit time is. In addition, the higher the purification setting is, the larger the consumption amount of the hypochlorous acid per unit time is. The description returns to FIG. 2.

Controller 500 integrates the consumption amounts of the hypochlorous acid per unit time. In other words, controller 500 calculates an integrated value of the consumption amounts of the hypochlorous acid based on the purification setting indicating a purification capability required of purification part 310, furthermore the air volume setting, and an elapsed time from previous timing at which the hypochlorous acid water has been previously supplied. When the integrated value of the consumption amounts of the hypochlorous acid becomes equal to or larger than a threshold value, controller 500 determines the supply of the hypochlorous acid water from electrolytic bath 200 to mixing bath 300. In other words, when the integrated value becomes equal to or larger than the supply amount of the hypochlorous acid at the previous timing, controller 500 causes third supply part 228 to supply next hypochlorous acid water. When the hypochlorous acid water is supplied, controller 500 clears the integrated value of the consumption amounts.

### (4-1-2) Second Condition

Controller 500 determines supply of hypochlorous acid water from electrolytic bath 200 to mixing bath 300 when the second condition is satisfied that the concentration of hypochlorous acid water in electrolytic bath 200 is lower than or equal to a second concentration lower than the first concentration. In the following, specific description will be given.

Count part 550 of controller 500 counts an elapsed time from timing at which generation of the hypochlorous acid water having the first concentration is completed in electrolytic bath 200.

The calculation part 560 of controller 500 calculates, based on count time counted by count part 550, a concentration decrease value indicating how much the first concentration of the hypochlorous acid water in electrolytic bath 200 is decreased. As described above, after generation of hypochlorous acid water having the first concentration in electrolytic bath 200, the hypochlorous acid in electrolytic bath 200 decreases and the concentration of the hypochlorous acid water decreases as time elapses in a state where the hypochlorous acid water and electrode part 210 are in contact with each other. Then, the concentration decrease value indicating how much the first concentration of the hypochlorous acid water in electrolytic bath 200 is decreased can be calculated based on the count time. Specifically, calculation part 560 increases the concentration decrease value as the count time becomes longer. The concentration decrease value indicating how much the first concentration of the hypochlorous acid water in electrolytic bath 200 corresponding to the count time varies with the volume of electrolytic bath 200 and the like, and can be calculated in advance by experiment or the like. As the concentration decrease value indicating how much the first concentration of the hypochlorous acid water in electrolytic bath 200 corresponding to the count time in the present exemplary embodiment, it is assumed, as an example, that the concentration decreases to half of the first concentration when the count time is 48 hours. Also in the present exemplary embodiment, it is assumed, as an example, that the concentration decreases to the second concentration when the count time is 144 hours. Here, the second concentration is a minimum hypochlorous acid water concentration as a hypochlorous acid water concentration required of hypochlorous acid water in mixing bath 300. Controller 500 supplies the hypochlorous acid water in electrolytic bath 200 to mixing bath 300 when the decreased hypochlorous acid water concentration in electrolytic bath 200 becomes less than or equal to the second concentration.

### (4-2) Supply Amount Control

First, description will be made of supply amount control of hypochlorous acid water to be supplied from electrolytic bath 200 to mixing bath 300 when the first condition is satisfied.

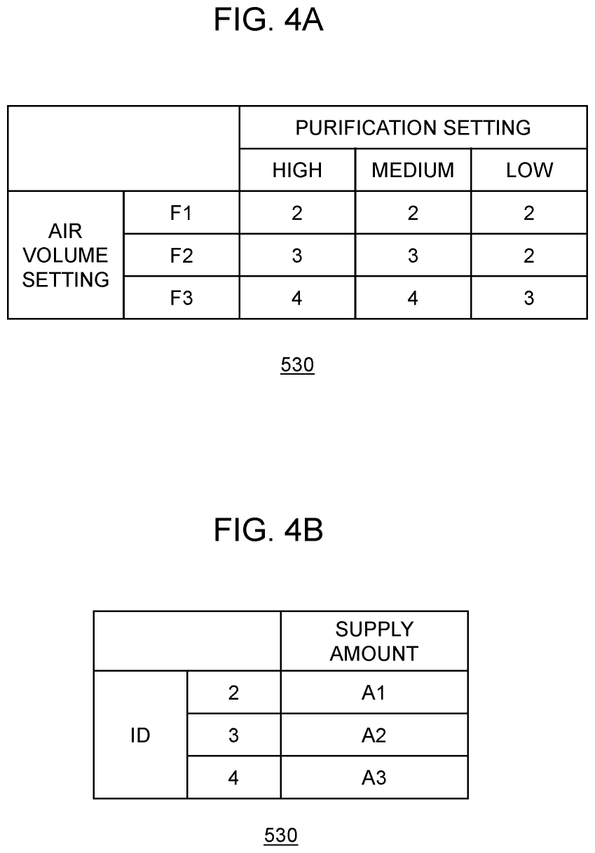

Controller 500 determines a supply amount of hypochlorous acid water to be supplied by third supply part 228 based on purification setting indicating a purification capability required of purification part 310 and air volume setting. Specifically, controller 500 receives the air volume setting and the purification setting from operation part 516. Controller 500 determines a supply amount of the hypochlorous acid water by third supply part 228 based on the purification setting and the air volume setting. FIGS. 4A and 4B show data structures of further tables stored in storage part 530. FIG. 4A is a table showing “IDs” associated with air volume setting “F1” to “F3” and the purification setting “high”, “medium”, and “low”. “ID” is the same as the “ID” shown in FIG. 3A. Controller 500 specifies “ID” based on the purification setting and the air volume setting.

FIG. 4B is a table showing a supply amount of hypochlorous acid water associated with “ID”. “ID=2” indicates a supply amount “A1”, “ID=3” indicates a supply amount “A2”, and “ID=4” indicates a supply amount “A3”. Here, A1<A2<A3. Controller 500 determines a supply amount of the next hypochlorous acid water based on a specified “ID”.

After determining the supply by the above-described processing and determining the supply amount, controller 500 adjusts a supply amount of the hypochlorous acid water supplied from third supply part 228 based on the concentration decrease value. Specifically, controller 500 increases the supply amount as the count time becomes longer. For example, in a case where the supply amount of the hypochlorous acid water is “A1” and the count time is 48 hours, since the hypochlorous acid water in electrolytic bath 200 has a concentration reduced to half of the first concentration, the supply amount of the hypochlorous acid water is to be doubled. Similarly, in a case where the count time is 96 hours, since the hypochlorous acid water in electrolytic bath 200 has a concentration reduced to one-fourth of the first concentration, the supply amount of the hypochlorous acid water is to be quadrupled. In this manner, controller 500 adjusts the supply amount of the hypochlorous acid water to be supplied from third supply part 228 based on the concentration decrease value corresponding to the count time.

This enables a desired amount of the hypochlorous acid to be supplied from electrolytic bath 200 to mixing bath 300. In other words, the concentration of the hypochlorous acid water in mixing bath 300 can be set to a desired concentration.

In addition, when the hypochlorous acid water is supplied from electrolytic bath 200 to mixing bath 300 for the first time after the power of space purification device 1000 is initially turned on, since the count time is 0, controller 500 does not adjust the supply amount of the hypochlorous acid water on supplying the hypochlorous acid water from third supply part 228.

In addition, when the power of space purification device 1000 is turned off, the count time at the time when the power is turned off may be stored in storage part 530. An example of storage part 530 at this time is a nonvolatile memory. For example, count part 550 can store the count time at the time of power-off by periodically storing the count time in storage part 530 when the power is turned on. In this case, after the power of the space purification device 1000 is turned on again, count part 550 restarts counting from the count time stored in storage part 530. In addition, a battery may be contained in space purification device 1000 to continue counting even when the power of space purification device 1000 is turned off. Specifically, a battery capable of supplying power to count part 550 when the power of space purification device 1000 is turned off may be further provided, so that count part 550 continues counting even when the power of space purification device 1000 is turned off. This enables count time to be accurately grasped even when the power of space purification device 1000 is turned off halfway. The power-off includes power-off due to a power failure or the like in addition to a case of intentional power-off by the user.

Next, description will be made of supply amount control of hypochlorous acid water to be supplied from electrolytic bath 200 to mixing bath 300 when the second condition is satisfied. Controller 500 supplies all the hypochlorous acid water in electrolytic bath 200 to mixing bath 300 when the decreased hypochlorous acid water concentration in electrolytic bath 200 becomes less than or equal to the second concentration. This enable hypochlorous acid generated in electrolytic bath 200 to be efficiently consumed in purification part 310.

Controller 500 causes third supply part 228 to supply hypochlorous acid water after determining the supply timing and determining the supply amount, and adjusting the supply amount as required by the above-described processing. The supply amount is controlled by, for example, a rotation speed and an operation time period of a pump motor in third supply part 228. The identified “ID” is used in the table of FIG. 3A for “(4-1) Supply timing control” when determining a next threshold value.

The device, the system, or a subject of the method in the present disclosure is provided with a computer. Execution of a program by this computer realizes the functions of the device, the systems, or the subject of the method in the present disclosure. The computer includes, as a main hardware configuration, a processor that operates according to the program. A type of the processor is not limited as long as the processor can realize the functions by executing the program. The processor includes one or a plurality of electronic circuits including a semiconductor integrated circuit (IC) or a large scale integration (LSI). The plurality of electronic circuits may be integrated on one chip or may be provided on a plurality of chips. The plurality of chips may be aggregated into one device or may be provided in a plurality of devices. The program may be recorded in a computer-readable non-transitory recording medium such as a read only memory (ROM). The program may be stored in advance in a recording medium, or may be supplied to the recording medium via a wide area communication network including the Internet.

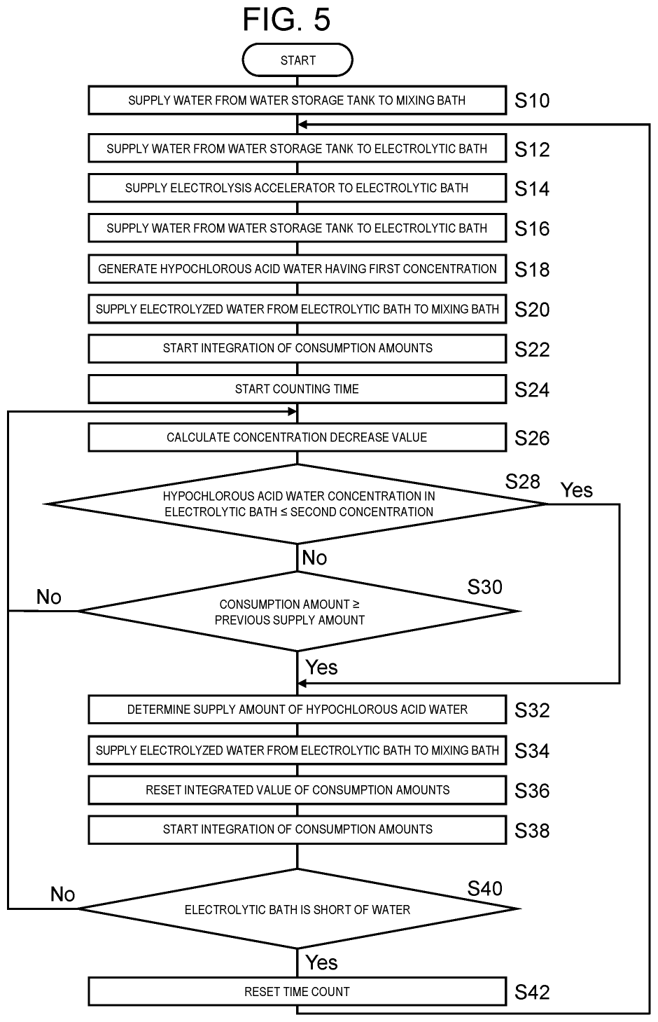

Operation of space purification device 1000 having the above configuration will be described. FIG. 5 is a flowchart showing a control procedure by space purification device 1000.

First, after the power of space purification device 1000 is initially turned on, water is supplied from water storage tank 100 to mixing bath 300 (S10).

Next, water is supplied from water storage tank 100 to electrolytic bath 200 by an amount smaller than a full capacity (S12).

Next, electrolysis accelerator 410 is supplied to electrolytic bath 200 (S14).

Next, water is supplied from water storage tank 100 to electrolytic bath 200 to the full capacity (S16).

Next, electrode part 210 executes electrolysis to generate hypochlorous acid water having the first concentration (S18).

Next, the hypochlorous acid water having the first concentration is supplied from electrolytic bath 200 to mixing bath 300 (S20). On this occasion, the supply amount is not adjusted by controller 500.

Furthermore, controller 500 starts integration of the consumption amounts of the hypochlorous acid derived based on the air volume setting and the purification setting (S22).

Next, count part 550 starts counting time (S24).

Next, calculation part 560 calculates a concentration decrease value from the first concentration, and calculates a current concentration of the hypochlorous acid water in electrolytic bath 200 from the concentration decrease value (S26).

Next, controller 500 determines whether or not the current hypochlorous acid water concentration in electrolytic bath 200 is higher than the second concentration (S28). When it is determined that the current hypochlorous acid water concentration in electrolytic bath 200 is higher than the second concentration (No in S28), controller 500 determines whether or not an integrated value of the consumption amounts of the hypochlorous acid is equal to or larger than the supply amount of the previous hypochlorous acid (S30).

When it is determined in Step S30 that the integrated value of the consumption amounts of the hypochlorous acid is not equal to or larger than the supply amount of the previous hypochlorous acid (No in S30), controller 500 returns the processing to Step S26.

When it is determined in Step S30 that the integrated value of the consumption amounts of the hypochlorous acid is larger than or equal to the previous supply amount (Yes in S30), controller 500 determines the supply of next hypochlorous acid water from electrolytic bath 200 to mixing bath 300 (S32). At this time, controller 500 determines a supply amount of the hypochlorous acid water based on the purification setting and the air volume setting. Furthermore, controller 500 adjusts the supply amount of the hypochlorous acid water based on the concentration decrease value.

Next, hypochlorous acid water of the supply amount determined and adjusted by controller 500 is supplied from electrolytic bath 200 to mixing bath 300 (S34).

Next, controller 500 resets the integrated value of the consumption amounts of the hypochlorous acid (Step S36).

Next, controller 500 starts integrating the consumption amounts of the hypochlorous acid (Step S38).

Next, controller 500 determines whether or not electrolytic bath 200 is short of water based on a detection result of water shortage float 260 (S40). When it is determined that electrolytic bath 200 is not short of hypochlorous acid water (No in S40), controller 500 returns the processing to Step S26. When it is determined that electrolytic bath 200 is short of hypochlorous acid water (Yes in S40), controller 500 resets time count after the generation of the first concentration (S42). Thereafter, the processing returns to Step S12.

When it is determined that the current hypochlorous acid water concentration in electrolytic bath 200 is less than or equal to the second concentration (Yes in S28), controller 500 determines the supply of next hypochlorous acid water from electrolytic bath 200 to mixing bath 300 (S32). At this time, controller 500 determines to supply all the hypochlorous acid water in electrolytic bath 200 to mixing bath 300. Thereafter, hypochlorous acid water of the supply amount determined by controller 500 is supplied from electrolytic bath 200 to mixing bath 300 (S34). At this time, since there is a possibility that water overflows from mixing bath 300, the hypochlorous acid water may be supplied in a plurality of times at intervals. In order to prevent water from overflowing from mixing bath 300, full water float 350 may be utilized.

In addition, when it is determined that the current hypochlorous acid water concentration in electrolytic bath 200 is less than or equal to the second concentration (Yes in S28), controller 500 may determine a supply amount of the hypochlorous acid water based on the purification setting and the air volume setting. Furthermore, controller 500 may adjust the supply amount of the hypochlorous acid water based on the concentration decrease value. As can be seen from the flowchart of FIG. 5, since when the hypochlorous acid water concentration in electrolytic bath 200 is less than or equal to the second concentration, the hypochlorous acid water is supplied from electrolytic bath 200 to mixing bath 300 until the hypochlorous acid water in electrolytic bath 200 runs out, all the hypochlorous acid water in electrolytic bath 200 is supplied to mixing bath 300 as a result.

According to the present exemplary embodiment, since the supply amount of hypochlorous acid water to be supplied from electrolytic bath 200 is adjusted based on the concentration decrease value, an appropriate amount of hypochlorous acid water can be supplied. In addition, since an appropriate amount of hypochlorous acid water can be supplied, active oxygen species can be released in an appropriate amount.

In addition, since a supply amount of hypochlorous acid water to be supplied from electrolytic bath 200 is determined based on purification setting and air volume setting, an appropriate amount of hypochlorous acid water can be supplied. In addition, since an appropriate amount of hypochlorous acid water can be supplied, active oxygen species can be released in an appropriate amount.

Furthermore, when an integrated value of consumption amounts of hypochlorous acid water becomes equal to or more than a supply amount of the hypochlorous acid water at the previous timing, supply of next hypochlorous acid water is determined, so that the hypochlorous acid water can be supplied at appropriate timing. In addition, since hypochlorous acid water can be supplied at appropriate timing, active oxygen species can be released in an amount suitable for a use environment.

When the decreased hypochlorous acid concentration in electrolytic bath 200 becomes less than or equal to the second concentration which is lower than the first concentration, the supply of all the hypochlorous acid water in electrolytic bath 200 is determined, so that the hypochlorous acid water can be supplied at appropriate timing. In addition, since all the hypochlorous acid water can be supplied at appropriate timing, a significant decrease in the hypochlorous acid concentration in mixing bath 300 can be prevented. In addition, it is possible to efficiently consume, in purification part 310, hypochlorous acid generated in electrolytic bath 200.

Furthermore, since the hypochlorous acid water having the first concentration is newly generated in electrolytic bath 200 after all the hypochlorous acid water in electrolytic bath 200 is supplied to mixing bath 300, the maintenance frequency of electrolytic bath 200 can be reduced. In addition, the concentration in electrolytic bath 200 can be constantly grasped by an elapsed time after generation of the first concentration.

In addition, since the concentration decrease value is increased as the count time is longer, the concentration decrease value can be accurately grasped.

An outline of one aspect of the present disclosure is as follows. Space purification device (1000) according to an aspect of the present disclosure includes: electrolytic bath (200) that stores water supplied from outside; electrode part (210) that is provided in electrolytic bath (200) and generates hypochlorous acid water having a first concentration from the water stored in electrolytic bath (200); mixing bath (300) that mixes the hypochlorous acid water and water to generate mixed water; hypochlorous acid water supply part (228) that supplies the hypochlorous acid water from electrolytic bath (200) to mixing bath (300); water supply part (138) that supplies water to mixing bath (300); air blower (518) that takes in air from an air suction port based on preset air volume setting; purification part (310) that brings the mixed water stored in mixing bath (300) into contact with the air sucked from the air suction port; and controller (500) that controls electrode part (210), hypochlorous acid water supply part (228), water supply part (138), and air blower (518). Controller (500) includes count part (550) that counts an elapsed time from timing at which generation of the hypochlorous acid water having the first concentration in electrolytic bath (200) is completed, and calculation part (560) that calculates, based on a count time counted by the count part, a concentration decrease value indicating how much the first concentration of the hypochlorous acid water in electrolytic bath (200) is decreased. Controller (500) is configured to adjust a supply amount of the hypochlorous acid water to be supplied from hypochlorous acid water supply part (228) based on the concentration decrease value.

Controller (500) may be configured to determine a supply amount of next hypochlorous acid by hypochlorous acid water supply part (228) based on purification setting indicating a purification capability required of purification part (310) and the air volume setting.

Controller (500) calculates an integrated value of consumption amounts of the hypochlorous acid based on purification setting indicating a purification capability required of purification part (310), the air volume setting, and an elapsed time after previous timing at which the hypochlorous acid water has been previously supplied. Controller (500) may be configured to cause hypochlorous acid water supply part (228) to supply next hypochlorous acid water when the integrated value becomes equal to or larger than the supply amount of the hypochlorous acid at the previous timing.

Controller (500) may be configured to supply all the hypochlorous acid water in electrolytic bath (200) to mixing bath (300) when decreased concentration of the hypochlorous acid water in electrolytic bath (200) becomes less than or equal to a second concentration which is lower than the first concentration.

Controller (500) may be configured to newly generate the hypochlorous acid water having the first concentration in electrolytic bath (200) after supplying all the hypochlorous acid water in electrolytic bath (200) to mixing bath (300).

Calculation part (560) may be configured to increase the concentration decrease value as the count time becomes longer.

The present disclosure has been described in the foregoing based on the exemplary embodiment. It is to be understood by those skilled in the art that this exemplary embodiment is an example, that various modifications in combinations of the components or the processing processes of the exemplary embodiment are possible, and that such modifications fall within the scope of the present disclosure.

### INDUSTRIAL APPLICABILITY

The space purification device according to the present disclosure is useful as an space purification device that removes bacteria, fungi, viruses, odors, or the like in air.

### REFERENCE MARKS IN THE DRAWINGS

100 water storage tank
110 water supply tank
112 lid
120 first pump
122 first water supply pipe
124 supply port
128 first supply part
130 second pump
132 second water supply pipe
138 second supply part (water supply part)
160 water shortage float
200 electrolytic bath
210 electrode part
220 third pump
222 third water supply pipe
224 fixed capacity container
226 third water supply pipe
228 third supply part (hypochlorous acid water supply part)
250 full water float
260 water shortage float
300 mixing bath
310 purification part
350 full water float
360 water shortage float
370 drainage float
400 electrolysis accelerator input part
404 input port
410 electrolysis accelerator
460 electrolyzed water generation part
500 controller
516 operation part
518 air blower
530 storage part
550 count part
560 calculation part
1000 space purification device

What is claimed is:

1. A space purification device comprising:
an electrolytic bath that stores water supplied from outside;
an electrode part that is provided in the electrolytic bath and generates hypochlorous acid water having a first concentration from the water stored in the electrolytic bath;
a mixing bath that mixes the hypochlorous acid water and water to generate mixed water;
a hypochlorous acid water supply part that supplies the hypochlorous acid water from the electrolytic bath to the mixing bath;
a water supply part that supplies the water to the mixing bath;
an air blower that takes in air from an air suction port based on preset air volume setting;
a purification part that brings the mixed water stored in the mixing bath into contact with the air sucked from the air suction port; and
a controller that controls the electrode part, the hypochlorous acid water supply part, the water supply part, and the air blower,
wherein
the controller includes:
a count part that counts an elapsed time from timing at which generation of the hypochlorous acid water having the first concentration in the electrolytic bath is completed, and
a calculation part that calculates, based on a count time counted by the count part, a concentration decrease value indicating how much the first concentration of the hypochlorous acid water in the electrolytic bath is decreased, and
the controller is configured to adjust a supply amount of the hypochlorous acid water to be supplied from the hypochlorous acid water supply part based on the concentration decrease value.

2. The space purification device according to claim 1, wherein the controller is configured to determine a supply amount of next hypochlorous acid water by the hypochlorous acid water supply part based on purification setting indicating a purification capability required of the purification part and the air volume setting.

3. The space purification device according to claim 1, wherein
the controller is configured to:
calculate an integrated value of consumption amounts of the hypochlorous acid based on purification setting indicating a purification capability required of the purification part, the air volume setting, and an elapsed time from previous timing at which the hypochlorous acid water has been previously supplied, and
cause the hypochlorous acid water supply part to supply next hypochlorous acid water when the integrated value becomes equal to or larger than the supply amount of the hypochlorous acid at the previous timing.

4. The space purification device according to claim 2, wherein
the controller is configured to:
calculate an integrated value of consumption amounts of the hypochlorous acid based on the purification setting, the air volume setting, and an elapsed time from previous timing at which the hypochlorous acid water has been previously supplied, and
cause the hypochlorous acid water supply part to supply next hypochlorous acid water when the integrated value becomes equal to or larger than the supply amount of the hypochlorous acid at the previous timing.

5. The space purification device according to claim 1, wherein the controller is configured to supply all the hypochlorous acid water in the electrolytic bath to the mixing bath when decreased concentration of the hypochlorous acid water in the electrolytic bath becomes less than or equal to a second concentration which is lower than the first concentration.

6. The space purification device according to claim 3, wherein the controller is configured to newly generate the hypochlorous acid water having the first concentration in the electrolytic bath after supplying all the hypochlorous acid water in the electrolytic bath to the mixing bath.

7. The space purification device according to claim 1, wherein the calculation part is configured to increase the concentration decrease value as the count time becomes longer.

8. The space purification device according to claim 1, further comprising a storage part that stores the count time at a time point of power-off of the space purification device, wherein
the count part resumes counting at the count time stored in the storage part after a power of the space purification device is turned on again which is after the time point of the power-off.

9. The space purification device according to claim 8, further comprising a battery capable of supplying power to the count part at the time of the power-off of the space purification device, wherein
the count part continues counting even at a time of the power-off of the space purification device.

10. The space purification device according to claim 4, wherein the controller is configured to newly generate the hypochlorous acid water having the first concentration in the electrolytic bath after supplying all the hypochlorous acid water in the electrolytic bath to the mixing bath.

11. The space purification device according to claim 5, wherein the controller is configured to newly generate the hypochlorous acid water having the first concentration in the electrolytic bath after supplying all the hypochlorous acid water in the electrolytic bath to the mixing bath.
